SEQUENCE LISTING (4 sequences)

SEQ ID NO: 1 - DNA, 21 bp, synthetic construct
  source 1..21: mol_type = other DNA
attcatagtctagttggaggt

SEQ ID NO: 2 - DNA, 20 bp, synthetic construct
  source 1..20: mol_type = other DNA
cctgaactgagagaatttga

SEQ ID NO: 3 - DNA, 24 bp, synthetic construct
  source 1..24: mol_type = other DNA
tgcttgcatcttgatttaattttg

SEQ ID NO: 4 - DNA, 25 bp, synthetic construct
  source 1..25: mol_type = other DNA
ggttcttggatytatgcggtattag

(19) United States
(12) Patent Application Publication
(10) Pub. No.: US 2025/0277186 A1
(43) Pub. Date: Sep. 4, 2025

(54) COMPOUND MICROBIAL INOCULUM WITH BACTERIOSTATIC EFFECT AND USE THEREOF IN PREPARATION OF YOGURT

(71) Applicant: SHIHEZI UNIVERSITY, Shihezi City (CN)

(72) Inventors: Yongqing Ni, Shihezi City (CN); Hailong Sun, Shihezi City (CN); Baolong Luo, Shihezi City (CN)

(21) Appl. No.: 19/211,375

(22) Filed: May 19, 2025

Related U.S. Application Data
(63) Continuation of application No. PCT/CN2024/128211, filed on Oct. 29, 2024.

(30) Foreign Application Priority Data
Dec. 10, 2023 (CN) 202311683560.7

(51) Int. Cl. C12N 1/20; A23C 9/123; A61K 35/744; A61P 1/12; C12R 1/01

(52) U.S. Cl. CPC C12N 1/205; A23C 9/1234; A61K 35/744; A61P 1/12; C12R 2001/01

(57) ABSTRACT

The present disclosure relates to the field of biotechnology, in particular to a compound microbial inoculum with bacteriostatic effect and use thereof in the preparation of yogurt. The compound microbial inoculum includes Lacticaseibacillusrhamnosus FMBL L23004 CNN and Lactiplantibacillus plantarum FMBL L23036 CNN, wherein the Lacticaseibacillus rhamnosus FMBL L23004 CNN was deposited on 26 Jun. 2023 at China Center for Type Culture Collection (CCTCC) under accession number CCTCC NO: M 20231099; and the Lactiplantibacillus plantarum FMBL L23036 CNN was deposited on 26 Jun. 2023 at CCTCC under accession number CCTCC NO: M 20231101. The compound microbial inoculum has the effect of preventing and/or treating diarrhea, and the compound microbial inoculum also has antibiotic sensitivity; and as a yogurt starter, the compound microbial inoculum allows yogurt obtained by fermentation to have higher probiotic activity.

### CROSS-REFERENCE TO RELATED APPLICATIONS

This application is a continuation of International Application No. PCT/CN2024/128211, filed on Oct. 29, 2024, which claims priority to Chinese Patent Application No. 202311683560.7, filed on Dec. 10, 2023. All of the aforementioned applications are incorporated herein by reference in their entireties.

### REFERENCE TO SEQUENCE LISTING

This application includes a Sequence Listing filed electronically as an XML file named “Sequence listing_ERICL-25007-USPT.xml”, created on May 7, 2025, with a size of 4,668 bytes. The Sequence Listing is incorporated herein by reference.

### TECHNICAL FIELD

The present disclosure relates to the field of biotechnology, in particular to a compound microbial inoculum with bacteriostatic effect and use thereof in the preparation of yogurt.

### BACKGROUND

Fermented milk is a product made from raw cow (sheep) milk or milk powder, which is sterilized and fermented by specific microorganisms, resulting in a decrease in pH value. Functional yogurt is a type of yogurt that can provide essential nutrients to the human body, meet people's needs for healthy food, and has obvious advantages in diet, with a wider range of application prospects. Dairy products are the best carrier for probiotics to enter the human body. There are many strains of bacteria used for fermenting probiotic yogurt. Studies have shown that strains of Lactobacillus, Streptococcus, Leuconostoc, and Bifidobacterium can all be used for fermenting yogurt, including common strains such as Lactobacillus delbrueckii, Lactobacillus acidophilus, Streptococcus cremoris, Streptococcus lactis, Leuconostoc oenos, Leuconostoc lactis, Leuconostoc mesenteroides and Leuconostoc mesenteroides subsp. cremoris, Bifidobacterium bifidum, Bifidobacterium breve, Bifidobacterium longum, Bifidobacterium infantis, Bifidobacterium adolescentis, and Pediococcus acidilactici.

In people and animals with diarrhea, the vast majority are found to be dysbacteriosis caused by pathogenic bacteria. On the one hand, invading pathogens inhibit the growth of normal bacteria, leading to a decrease in the number of beneficial bacteria in the gastrointestinal tract; and on the other hand, toxic substances produced by pathogens further cause abnormal intestinal function and immune response, which results in the occurrence of diarrhea. Bacterial diarrhea is a global health issue, especially in developing countries, where enteropathogenic bacteria are a major cause of infectious diarrhea. At present, Escherichia coli, Shigella, Salmonella, Campylobacter, Clostridium difficile, and Aeromonas are the main pathogens causing diarrhea.

Probiotics can promote the growth and reproduction of beneficial bacteria in the intestinal tract of a host, regulate the immune system of the host, promote the absorption of beneficial nutrients in the intestinal tract, improve digestive system efficiency, reduce intestinal inflammation, and reduce the absorption of intestinal toxins, thereby improving intestinal health and preventing the occurrence of intestinal diseases. Probiotics can treat diarrhea caused by pathogens by maintaining or improving the balance of the intestinal microbiota, and their mechanism may be related to inhibiting the colonization of harmful bacteria by competing for nutrients and producing antibacterial compounds. In addition, Lacticaseibacillus rhamnosus LGG can regulate the maturation and differentiation of dendritic cells and the secretion of inflammatory factors, thereby preventing diarrhea caused by rotavirus. In summary, the beneficial effects of probiotics on diarrhea are related to strains and their dosage, and the selection and use of the best probiotics for treating diarrhea need to be determined through more clinical trials.

According to the present disclosure, excellent strains having the ability to resist diarrheal pathogens are screened out, a multi-strain combination test is carried out, and thus a pair of compound microbial inocula with good anti-diarrhea capability are screened out and further applied to yogurt production; and the influence of a probiotic strain combination on yogurt quality is analyzed by optimizing various yogurt indexes of the traditional starter strains, thus laying a foundation for the research and development of anti-diarrhea functional fermented dairy products.

### SUMMARY

The primary object of the present disclosure is to provide a compound microbial inoculum with bacteriostatic effect. The compound microbial inoculum includes Lacticaseibacillus rhamnosus FMBL L23004 CNN and Lactiplantibacillus plantarum FMBL L23036 CNN, where the Lacticaseibacillus rhamnosus FMBL L23004 CNN was deposited on 26 Jun. 2023 at China Center for Type Culture Collection (CCTCC) under accession number CCTCC NO: M 20231099; and the Lactiplantibacillus plantarum FMBL L23036 CNN was deposited on 26 Jun. 2023 at CCTCC under accession number CCTCC NO: M 20231101.

The second object of the present disclosure is to provide use of the compound microbial inoculum in the preparation of drugs for inhibiting pathogenic bacteria.

Preferably, the pathogenic bacteria include one or more of Escherichia coil EPEC, Escherichia coil ETEC, Salmonella enterica subsp. enterica serovar Typhimurium, Escherichia coil EHEC, Listeria monocytogenes, and Salmonella enterica subsp. Enterica.

The third object of the present disclosure is to provide use of the compound microbial inoculum in the preparation of drugs for preventing and/or treating diarrhea.

The fourth object of the present disclosure is to provide use of the compound microbial inoculum in the preparation of food, food additives or health care products.

The fifth object of the present disclosure is to provide use of the compound microbial inoculum in the preparation of yogurt or yogurt starter.

The sixth object of the present disclosure is to provide yogurt fermented by the compound microbial inoculum.

The beneficial effects of the present disclosure are as follows: the present disclosure provides a compound microbial inoculum with bacteriostatic effect, which includes Lacticaseibacillus rhamnosus FMBL L23004 CNN and Lactiplantibacillus plantarum FMBL L23036 CNN. The compound microbial inoculum has the effect of preventing and/or treating diarrhea, and the compound microbial inoculum also has antibiotic sensitivity. As a yogurt starter, the compound microbial inoculum allows yogurt obtained by fermentation to have suitable acidity and higher water holding capacity, as well as better texture characteristics, and have the typical flavor characteristics of yogurt; and the yogurt fermented by the compound microbial inoculum has higher probiotic activity.

### BRIEF DESCRIPTION OF THE DRAWINGS

FIG. 1 shows count of viable bacteria during yogurt storage.

FIG. 2A shows standard curve for bacterial count determination of Lacticaseibacillus rhamnosus FMBL L23004 CNN.

FIG. 2B shows standard curve for bacterial count determination of Lactiplantibacillus plantarum FMBL L23036 CNN.

FIG. 3 shows number of viable bacteria of Lactiplantibacillus plantarum and Lacticaseibacillus rhamnosus during yogurt storage.

### DETAILED DESCRIPTION OF THE EMBODIMENTS

The present disclosure will be further described below with reference to the accompanying drawings and specific embodiments, so as to enable those skilled in the art to better understand and implement the present disclosure, but the illustrated embodiments are not intended to limit the present disclosure.

Titrable acidity represents the total amount of all acidic substances in a yogurt system, while the pH of yogurt reflects the concentration of H⁺ in the system, both of which are related and different.

Acidity has a crucial impact on the processing cycle, production efficiency and flavor of yogurt, while high acidity will adversely affect its water retaining capacity and viscosity. Therefore, controlling acidity has become an essential skill in the production of yogurt, and it is also the key to ensuring product quality.

The water holding capacity of yogurt means that the protein in yogurt can retain water and form a gel-like network to maintain the consistency of yogurt. When the water holding capacity is weak, the protein network becomes loose and whey discharge will be observed, resulting in a poorer texture of yogurt. Therefore, the water holding capacity of yogurt can reflect the density of the gel network and the texture of yogurt, which is an important indicator for testing the quality of yogurt. Due to the influence of additives, temperature, pH and other factors, the water holding capacity of yogurt will be affected, thereby affecting the texture and taste of yogurt. Therefore, during production of yogurt, it is necessary to pay attention to controlling these factors to ensure the water holding capacity and quality of yogurt.

There are many substances that affect the good flavor of yogurt, most studies believe that the main flavor components of yogurt are diacetyl and acetaldehyde, so that the determination of diacetyl and acetaldehyde contents in yogurt is essential, which is also an important factor in the evaluation of yogurt flavor quality. The peak period for yogurt to produce flavor substances is generally after the end of fermentation, so it is necessary to after-ripen yogurt for about 1 d in order to make it have a good taste and flavor. Therefore, the contents of diacetyl and acetaldehyde in yogurt are determined after 1 d of yogurt after-ripening.

Yogurt is highly favored by consumers due to its unique hardness, dense texture, sweet aroma of dairy products, and moderate acidity. Therefore, viscosity is one of the most important qualities of yogurt. Yogurt with different strains of bacteria has different texture characteristics due to differences in the added strains and strain specificity. It is known that the texture of yogurt prepared by different combinations of strains is different. The hardness of yogurt is an important feature of its gel structure. The greater the hardness, the more conducive to the storage and transportation of yogurt. Compared with the yogurt in a control group, the sample yogurt is more prominent in terms of hardness, and the consistency of the yogurt is higher than that of the control group. The higher the consistency, the better the rheological properties of the yogurt. Cohesion reflects the degree of aggregation inside yogurt, and the cohesion of the sample is good. The viscosity index reflects the degree to which yogurt is affected by temperature changes, and the higher the viscosity index, the less affected the consistency is by temperature.

Alcohols are important components of yogurt flavor, mainly produced by lactose fermentation, amino acid metabolism and aldehyde conversion, which can endow yogurt with unique aroma and mellow taste. Lactose fermentation is the main way to produce alcohols. The alcohols produced by lactose fermentation can effectively enhance the flavor of yogurt, so that the yogurt has a rich aroma and mellow taste. In addition, amino acid metabolism is also an important way to produce alcohols. The alcohols produced by amino acid metabolism can adjust the sour taste of yogurt and make the taste of yogurt more rounded. Moreover, the conversion of aldehydes is also a way to produce alcohols. The alcohols obtained after the conversion of aldehydes can add a touch of sweetness to the yogurt, making the yogurt more delicious.

Acids are mainly produced by lactic acid bacteria fermentation and lactose fermentation. These processes can break down proteins, carbohydrates and fats, thus changing the taste and structure of food. Acids play an important role in human health, which can help the digestive system digest food better, promote the digestion and absorption of food, and thus improve the nutrition intake of human body.

Esters are mainly produced through esterification reactions, and they have pleasant sweetness and fruity aroma. They are one of the important components of yogurt and also an important substance that improves the taste of yogurt. The content of esters will affect the flavor and taste of yogurt. Aldehydes are mainly produced by lactose fermentation and bacterial fermentation, which may undergo aromatic substitution reactions to form the main aromatic substances in yogurt, and are also one of the important components of lactic acid beverages and yogurt. Acetaldehyde is a typical flavor substance in yogurt, and aldehydes can improve the taste and flavor of yogurt, thereby enhancing its quality. Ketones are mainly derived from raw milk, bacterial fermentation, citric acid fermentation, and the like, and their unique flavor plays an important role in yogurt. The ketones in raw milk mainly come from the action of lactic acid bacteria and lactic acid glycosidase. After fermentation, lactic acid bacteria will produce lactic acid, while lactic acid glycosidase will produce ethyl lactate, which will be further converted into ketones. Citric acid fermentation is also a common method for producing ketones. During the fermentation process, citric acid will be oxidized to form ketones such as acetolactic acid and ethyl acetolactate. Bacterial fermentation can also produce ketones.

In the following embodiments, the abbreviations and full names of strains are shown in the table below.

Pathogenic bacteria and medium

| Strain | Medium |
|---|---|
| Escherichia coil EPEC O127:K63 CICC 10411 | Nutrient agar |
| | medium |
| Escherichia coil ETEC O78:K80 CICC 10421 | Nutrient agar |
| | medium |
| Salmonella enterica subsp. enterica serovar | Nutrient agar |
| Typhimurium CICC 10420 | medium |
| Escherichia coil EHEC O157:H7 CICC 21530 | Nutrient agar |
| | medium |
| Listeria monocytogenes CGMCC 1.9136 | PYG broth |
| | medium |
| Salmonella enterica subsp. Enterica CGMCC 1.10754 | TSA medium |

### Embodiment 1: Anti-Diarrhea Effect of Compound Microbial Inoculum

### 1. Strains

A compound microbial inoculum includes Lacticaseibacillus rhamnosus FMBL L23004 CNN and Lactiplantibacillus plantarum FMBL L23036 CNN, where the Lacticaseibacillus rhamnosus FMBL L23004 CNN was deposited on 26 Jun. 2023 at China Center for Type Culture Collection (CCTCC) under accession number CCTCC NO: M 20231099; and the Lactiplantibacillus plantarum FMBL L23036 CNN was deposited on 26 Jun. 2023 at CCTCC under accession number CCTCC NO: M 20231101.

2. Experimental methods
(1) Sugar metabolism experiment

The strains were tested for the metabolism of lactose, glucose, fructose and galactooligosaccharides. 1 mL of a bacteria solution which had been activated for two generations and reached a log phase was centrifuged at 10000 rpm for 5 min to collect bacterial cells, washed twice with sterilized physiological saline and then resuspended to produce a bacterial suspension. Lacticaseibacillus rhamnosus FMBL L23004 CNN and Lactiplantibacillus plantarum FMBL L23036 CNN were mixed together according to a ratio of 1:1, and separately inoculated into MRS mediums with different sugars as the sole carbon source at a 2% inoculation rate. Single strains were respectively fermented as control groups, anaerobic cultivation was carried out at 37° C. for 24 h, and the OD₆₀₀ was determined. Three parallel experiments were set up.

### (2) Bacteriostatic Properties

The bacteriostatic ability of the strains was determined by an Oxford cup method. Escherichia coil EPEC, Escherichia coil ETEC, Salmonella enterica subsp. enterica serovar Typhimurium, Escherichia coil EHEC, Listeria monocytogenes, and Salmonella enterica subsp. Enterica were used as indicator bacteria, 100 μL (about 10⁷ CFU/mL) of the indicator bacteria were coated on surfaces of the corresponding solid media, sterile Oxford cups were evenly placed on a plate at equal intervals, and 200 μL of mixed bacterial suspension (combination 1:1) was added to the Oxford cups. After pre-diffusion in a refrigerator at 4° C. for 6 h, the plate was placed in a 37° C. constant temperature incubator and cultured for 24 h. The diameter of an inhibition zone was observed and measured three times in parallel to take the average value.

### 3. Experimental Results

### (1) Sugar Metabolism Experiment

As shown in Table 1, compared with single bacteria, the compound microbial inoculum has better utilization ability for glucose, fructooligosaccharide, and galactooligosaccharides, with a higher utilization ability for lactose than the single bacterium Lacticaseibacillus rhamnosus FMBL L23004 CNN, and a slight lower utilization ability for lactose than the single bacterium Lactiplantibacillus plantarum FMBL L23036 CNN.

TABLE 1
Utilization of lactose, glucose, fructose and
galactose by compound microbial inoculum

| | Lacticaseibacillus | | |
|---|---|---|---|
| | rhamnosus FMBL | | |
| | L23004 CNN + | | Lactiplantibacillus |
| | Lactiplantibacillus | Lacticaseibacillus | plantarum |
| | plantarum FMBL | rhamnosus FMBL | FMBL L23036 |
| | L23036 CNN | L23004 CNN | CNN |
| Glucose | 1.588 ± 0.047^{a} | 0.987 ± 0.001^{c} | 1.408 ± 0.001^{b} |
| Lactose | 1.212 ± 0.020^{a} | 1.143 ± 0.001^{b} | 1.402 ± 0.002^{a} |
| Fructooligosaccharide | 1.579 ± 0.023^{a} | 1.432 ± 0.002^{b} | 1.351 ± 0.001^{c} |
| Galactooligosaccharides | 1.581 ± 0.032^{a} | 1.068 ± 0.002^{c} | 1.387 ± 0.001^{b} |

Note:
Different letters within the same row represent significant differences (P < 0.05)

### (2) Bacteriostasis Experiment

As shown in Table 2, compared with single strains, the composite strain showed enhanced inhibitory activity against Escherichia coil EHEC, diarrheal E. coli, Salmonella typhimurium, and Salmonella enterica serovar Enteritidis. Compared with the single strain of Lacticaseibacillus rhamnosus FMBL L23004 CNN, the compound microbial inoculum showed enhanced bacteriostatic activity except for Escherichia coil ETEC; and compared with the single strain of Lactiplantibacillus plantarum FMBL L23036 CNN, except for Listeria monocytogenes, the compound microbial inoculum showed enhanced bacteriostatic activity against the other five indicator bacteria.

TABLE 2
Bacteriostatic activity of different strains

| | Indicator/mm | | | | | |
|---|---|---|---|---|---|---|
| | | | | | LS1- | SM1- |
| strains | 21530 | 10411 | 10421 | 10420 | PYG | TSA |
| Lacticaseibacillus | 26.01 ± | 24.78 ± | 17.64 ± | 20.07 ± | 9.85 ± | 18.83 ± |
| rhamnosus FMBL | 0.75^{a} | 0.31^{a} | 0.98^{b} | 0.07^{a} | 0.34^{b} | 0.46^{a} |
| L23004 CNN | | | | | | |
| Lactiplantibacillus | | | | | | |
| plantarum FMBL | | | | | | |
| L23036 CNN | | | | | | |
| Lacticaseibacillus | 9.48 ± | 20.45 ± | 22.66 ± | 19.96 ± | 9.44 ± | 11.77 ± |
| rhamnosus FMBL | 0.22^{c} | 0.19^{b} | 0.74^{a} | 0.13^{a} | 0.39^{b} | 0.22^{c} |
| L23004 CNN | | | | | | |
| Lactiplantibacillus | 12.67 ± | 13.81 ± | 17.37 ± | 15.39 ± | 14.12 ± | 16.66 ± |
| plantarum FMBL | 0.27^{b} | 0.13^{c} | 2.48^{b} | 0.21^{b} | 0.56^{a} | 0.19^{b} |
| L23036 CNN | | | | | | |

Note:
Different letters within the same column represent significant differences (P < 0.05)

### Embodiment 2: Antibiotic Sensitivity

### 1. Experimental Method

After two strains were cultured anaerobically at 37° C. at a 2% inoculation rate for 24 h, 100 μL (about 1×10⁷ cfu/mL) of a bacteria solution of each strain was coated on the surface of an MRS solid medium, and then drug-sensitive paper (purchased from Oxoid Company, UK) was lightly pressed tightly in the middle of each of the mediums; and after anaerobic culture was carried out at 37° C. for 24 h, the bacteriostatic condition of the drug-sensitive paper was observed, and the diameter of the inhibition zone was measured. The information of the drug-sensitive paper is shown in Table 3.

TABLE 3
Information of drug-sensitive paper

| Antibiotic name | Abbreviation | Content | |
|---|---|---|---|
| Ampicillin | AMP | 10 | μg/disc |
| Oxacillin | OX | 1 | μg/disc |
| Cephaloridine | CTX | 30 | μg/disc |
| Clindamycin | DA | 2 | μg/disc |
| Polymyxin | CT | 10 | μg/disc |
| Norfloxacin | NOR | 10 | μg/disc |
| Teicoplanin | TEC | 30 | μg/disc |
| Vancomycin | VA | 30 | μg/disc |
| Gentamicin | CN | 120 | μg/disc |
| Amikacin | AK | 30 | μg/disc |
| Minocycline | MH | 30 | μg/disc |
| Kanamycin | K | 30 | μg/disc |
| Ciprofloxacin | CIP | 5 | μg/disc |
| Erythromycin | E | 15 | μg/disc |
| Rifampicin | RD | 5 | μg/disc |
| Penicillin | P | 10 | μg/disc |
| Streptomycin | S | 300 | μg/disc |
| Chloramphenicol | C | 30 | μg/disc |
| Tetracycline | TE | 30 | μg/disc |
| Hydroampicillin | AML | 10 | μg/disc |

### 2. Experimental Result

It is known that the two strains are resistant to gentamicin, amikacin, vancomycin, teicoplanin, norfloxacin, polymyxin, kanamycin, and ciprofloxacin, but sensitive to penicillin, ampicillin, cephaloridine, clindamycin, tetracycline, minocycline, chloramphenicol, rifampicin, and amoxicillin.

TABLE 4
Antibiotic sensitivity of two strains

| | Lacticaseibacillus | Lactiplantibacillus | | Lacticaseibacillus | Lactiplantibacillus |
|---|---|---|---|---|---|
| | rhamnosus | plantarum | | rhamnosus | plantarum |
| | FMBL L23004 | FMBL L23036 | | FMBL L23004 | FMBL L23036 |
| | CNN | CNN | | CNN | CNN |
| Penicillin | S | S | Rifampicin | S | S |
| Ampicillin | S | S | Vancomycin | R | R |
| Oxacillin | I | R | Teicoplanin | R | R |
| Cephaloridine | S | S | Norfloxacin | R | R |
| Clindamycin | S | S | Polymyxin | R | R |
| Gentamicin | R | R | Amoxicillin | S | S |
| Amikacin | R | R | Kanamycin | R | R |
| Tetracycline | S | S | Streptomycin | I | R |
| Minocycline | S | S | Ciprofloxacin | R | R |
| Chloramphenicol | S | S | Erythromycin | S | I |

Note:
R: resistant;
I: intermediately sensitive;
S: sensitive

### Embodiment 3: Preparation of Yogurt by Fermentation with Compound Microbial Inoculum

### 1. Experimental Method

### (1) Preparation of Yogurt

Through aseptic operation, a strain combination was inoculated into 100 mL of sterilized whole milk at a 2% inoculation rate, and thoroughly stirred to ensure uniform mixing; then, the obtained mixture was cultured at 42° C. until it became a curd state; and finally, the product was refrigerated in a refrigerator at 4° C. for 24 h of ripening.

### (2) Sensory Evaluation of Yogurt

The sensory evaluation of yogurt is shown in Table 5.

TABLE 5
Scoring criteria for sensory evaluation

| | Whey | | | | |
|---|---|---|---|---|---|
| | discharge | | | | |
| Score | phenomenon | Color | Taste | Flavor | Viscosity |
| 16-20 | Without | Milky | Moderate | Having the | Moderate |
| | whey | white | sweet and | taste and | viscosity |
| | | | sour | smell of | |
| | | | | yogurt | |
| 11-15 | Basically | White | Sour | Having | Thick and |
| | without | with | | astringent | hard |
| | whey | yellowish | | taste | |
| | | tint | | | |
| 6-10 | A little whey | Light | Insufficient | Having bitter | Thin |
| | | yellow | acidity | taste | |
| 0-5 | Large | Yellow | Strange | Not having | Too thin |
| | amount of | | odor | the taste and | or too |
| | whey | | | smell of | hard |
| | | | | yogurt | |

### (3) Determination of Acidity

The acidity of the fermented yogurt upon after-ripening was determined by titration. First, 10 g of yogurt was weighed in a beaker, 20 mL of distilled water was added, and the obtained mixture was mixed evenly; and 2-3 drops of a 0.5% phenolphthalein indicator was added dropwise and mixed evenly, a 0.1 mo/L NaOH standard solution was used for titrating, and the obtained mixture was mixed well while titrating until the solution turns pale pink and does not fade within 30 s. The value of acidity (° T) is the number of milliliters of the NaOH standard solution consumed multiplied by 10.

### (4) PH Measurement

The pH value of a yogurt sample was measured with a pH meter, and the sample was mixed with a glass rod to ensure an accurate measurement result.

### (5) Determination of Water Holding Capacity

A 50 mL centrifuge tube was selected for use, and the centrifuge tube was weighed, with the mass recorded as m₁; about 10 g of yogurt was taken and put into the centrifuge tube, with the mass of the centrifuge tube and yogurt recorded as m₂; after the centrifuge tube was centrifuged at 5000 r/min for 30 min at room temperature, the supernatant was poured out, and the centrifuge tube was turned upside down for 10 min, with the mass recorded as m₃; and the calculation formula of the water holding capacity (WHC) of the yogurt is as follows:

$$WHC = ( {m}_{3} - {m}_{1 )} / ( {m}_{2} - {m}_{1} )$$

(6) Determination of acetaldehyde content in Yogurt

The sample was stirred with 16% TCA in a certain proportion, the obtained mixture was centrifuged at 3500 r/min for 10 min, 25 mL of supernatant was placed in an iodine volumetric flask, 5 mL of 1% NaHSO₃ was added, and the iodine volumetric flask was shaken and placed in the dark for 1 h; then, 1 mL of 1% starch was added, and the obtained mixture was titrated with a 0.1 mol/L iodine solution until almost no color was observed; a 0.01 mol/L iodine solution was then added until the mixture was light blue; 20 mL of a 1mol/L sodium bicarbonate solution was added, and the obtained mixture was stirred while being shaken, and then was continued to be titrated with a 0.01 mol/L iodine standard solution until it appeared light blue again; and the volumes of the iodine solutions used were measured, and three parallel experiments were conducted.

Acetaldehyde content calculation formula:

$$Acetaldehyde ( g / mL ) = [ ( {V}_{1} - {V}_{2} ) C \times 0.0 \; 22 ] / 25$$

V₂ represents the volume (mL) of an I₂ standard solution consumed by blank control titration; V₁ represents the volume (mL) of an I₂ standard solution consumed by sample titration; C represents the concentration (mol/L) of an I₂ standard solution; 25 represents the weight (mL) of an acetaldehyde sample; and 0.022 represents the basic unit (g) of acetaldehyde chemical reaction.

### (7) Determination of Diacetyl Content in Yogurt

10 g of fermented yogurt obtained upon after-ripening was taken and put into a 50 mL centrifuge tube, 10 mL of a 16% TCA solution was added and mixed evenly, and the obtained mixture was centrifuged at 3500 r/min for 10 min at room temperature; 5 mL of supernatant was absorbed, and 0.25 mL of a 1 g/100 mL o-phenylenediamine solution was added separately, shaken evenly and placed in the dark for 30 min; then, 1.0 mL of a 4.0 mol/L HCl solution was added to terminate the reaction; and the absorbance at a wavelength of 335 nm was measured with a quartz cuvette. Three parallel tests were performed on each sample. The mass concentration of butanedione in the sample to be tested could be obtained by comparing the butanedione standard curve (standard curve reference).

### (8) Yogurt Texture Determination and Principal Component Analysis

Textural analysis was carried out on the samples obtained upon after-ripening, mainly referring to the method described by Changkun Li et al. with slight modifications for determination. An A/BE probe with a diameter of 35 mm was used in a 200 ml glass container (with a diameter of 64 mm and a height of 70 mm), and the speeds before, during and after measurement were 1.0 mm/s, 1.0 mm/s and 5.0 mm/s, respectively; and the parameters of penetration distance 20 mm and surface trigger force 10 g were tested.

(9) Determination of the number of Viable Bacteria during the Preservation of Yogurt

The number of Streptococcus thermophilus and the total number of lactic acid bacteria in yogurt were determined by a plate count method, and the number of Lactiplantibacillus plantarum and the number of Lacticaseibacillus rhamnosus in yogurt were determined by a real-time fluorescence quantitative method.

### A. Lactic Acid Bacteria Plate Count

According to the standard GB 4789.35-2016, about 1 g of the sample was taken and diluted 10 times with 0.85% sterile physiological saline; 0.9 mL of sterile physiological saline with a concentration of 8.5 g/L was added to 0.1 mL of diluent, and then the diluent was diluted to 10-7; two consecutive suitable degrees of dilution were selected, with 100 μL of diluent taken at each degree of dilution; and the diluents were coated on an M17 plate (for S. thermophilus) and an MRS plate (for total number of lactic acid bacteria) respectively; two parallel tests were performed at each degree of dilution, and anaerobic culture was carried out at 37° C. for 48 h; the number of colonies on the plates was counted, and the number of lactic acid bacteria was obtained by combining the degrees of dilution with the sampling amount; and the data result was expressed as CFU/g.

Extraction of DNA from Yogurt

The extraction of DNA from yogurt samples was carried out according to the operating steps of a kit. Before DNA extraction, the yogurt samples should be diluted, and the DNA products should be stored at −20° C. to prevent degradation.

### B. Real-Time Fluorescence Quantitative PCR Method for Counting Lactic Acid Bacteria

First, a standard curve was made, and the extracted DNA was diluted 10 times until it was diluted to 10⁵ times; and then, a counting standard curve of various lactic acid bacteria, that is, a relationship curve between the logarithmic value of lactic acid bacteria and Ct value, was made with 10¹-10⁵ times of nucleic acid diluent as a template. The reaction system and reaction conditions of the real-time fluorescence quantitative PCR are shown in Table 6. The specific primer sequences and amplification product lengths for Lactiplantibacillus plantarum and Lacticaseibacillus rhamnosus are shown in Table 7.

After the DNA of a yogurt sample was extracted by the kit, real-time fluorescence quantitative PCR was carried out according to the reflection system and conditions of the standard curve, and the obtained Ct value was substituted into the corresponding standard curve. The corresponding Lactobacillus number in the yogurt sample was calculated by combining the sampling amount with the dilution factor of the yogurt sample DNA. The result was expressed as Log CFU/mL.

TABLE 6
Real-time fluorescence quantitative PCR

| Real-time | |
|---|---|
| fluorescence | |
| quantitative PCR | Conditions |
| Reaction | DNA template 1.5 μL, each of upstream and |
| system | downstream primers 0.2 μL, ddH₂O 8.1 μL, Mixture |
| | 10 μL |
| Procedural | Pre-denaturation at 95° C. for 30 s, denaturation at 95° |
| conditions | C. for 10 s, annealing at 55° C. for 30 s, extension at |
| | 72° C. for 25 s, a total of 40 cycles |

TABLE 7
Specific primers for different lactic
acid bacteria

| | | | Product |
|---|---|---|---|
| | | | length |
| Strain | Primer name | Sequence 5-3 | (bp) |
| L. | L. plantarum-F | ATTCATAGTCTAGTTG | 248 |
| plantarum | (SEQ ID NO. 1) | GAGGTCCTGAACTGAG | |
| | L. plantarum-R | AGAATTTGA | |
| | (SEQ ID NO. 2) | | |
| L. | L. rhamnosus-F | TGCTTGCATCTTGATT | 122 |
| rhamnosuS | (SEQ ID NO. 3) | TAATTTTG | |
| | L. rhamnosus-R | GGTTCTTGGATYTATG | |
| | (SEQ ID NO. 4) | CGGTATTAG | |

### (10) Determination of Flavor Substances in Yogurt

### A. Sample Pretreatment and SPME Extraction Method

The volatile components in fermented milk were analyzed by solid-phase microextraction (SPME). Firstly, an extraction head was put into gas chromatography (GC) and placed at 245-255° C. for 2 h. The extraction head needs to be aged for another 10 min before extraction and every time a sample is extracted, which can remove the residual substances in the extraction head and ensure that it will not affect the determination of volatile components in the next sample. 5 g of a sample was taken and added into a 20 mL sample bottle, 1 g of NaCL and 1 μL of 2-methyl-3-heptanone were added, and the obtained mixture was stabilized at 50° C. for 20 min; when equilibrium was reached, a 50/30 μm DVB/CAR/PDMS extraction head was inserted into a sample inlet for aging treatment for 10 min, then a 50/30 μm DVB/CAR/PDMS extraction head was immediately inserted into the sample inlet for aging treatment for 10 min, and the extraction head was immediately placed into the sample bottle for adsorbing at 50° C. for 30 min; and the extraction head was immediately inserted into an sample inlet end after collection for desorbing at 250° C. for 5 min, the collected volatile substances were released at a starting end of the column, and GC-MS analysis was carried out.

### B. GC-MS Detection Conditions

GC conditions: HP-INNOWAX column, He gas, flow rate: 1.0 ml/min, line speed: 40 cm/s, split ratio: 1:10. The sample injection temperature was set to be 240° C. Programmed heating was employed, an initial temperature was set to be 40° C. for 10min, then it was raised to 140° C. at a rate of 4-5° C./min for 5 min, and finally, it was gradually raised to 250° C. at a rate of 10° C./min for 10 min. The temperature of a transmission line was set at 250° C.

MS conditions: An electric ion source worked at 150° C. in an electron shock mode, the voltage was 70 eV, the ion source temperature was 230° C., and the scanning range was 40-400 m/z. All yogurt samples were recorded by mass spectrometry, and scanning was performed for 5 times, without solvent delay. NISI library was used for identification.

### 2. Experimental Results

### (1) Determination of Physicochemical Properties of Yogurt

The pH value of combined yogurt was lower than that of control group, and the acidity thereof was higher than that of control group. The sensory score of the combined yogurt was 88.77, the acidity thereof was 96° T, the pH value thereof was 4.46, and the water holding capacity thereof was 67%.

TABLE 8
The pH value, acidity, water holding capacity and sensory
evaluation results of different combined yogurt

| | | | Water | |
|---|---|---|---|---|
| | | | holding | Sensory |
| | pH | Acidity | capacity | score |
| 6-4 + D-2 + Lacticaseibacillus | 4.46 ± | 96 ± | 0.67 ± | 88.77 ± |
| rhamnosus FMBL L23004 | 0.02 | 2.05 | 0.02 | 5.6 |
| CNN + Lactiplantibacillus | | | | |
| plantarum FMBL L23036 CNN | | | | |
| 6-4 + D-2 | 4.78 ± | 84 ± | 0.54 ± | 84.69 ± |
| | 0.02 | 4.5 | 0.01 | 4.16 |
| P-value | <0.01 | <0.01 | <0.01 | <0.01 |

### (2) Determination of Acetaldehyde and Diacetyl Contents

Generally, the peak period of producing flavor substances in yogurt is after fermentation, so it is necessary to after-ripen yogurt for about 1 d in order to make it have a good taste and flavor. Therefore, the contents of diacetyl and acetaldehyde in yogurt were determined after 1 day of after-ripening. The acetaldehyde content and diacetyl content of yogurt prepared from the compound microbial inoculum were both higher than those of a control group, which were 17.60 mg/L and 5.65 mg/L, respectively. When the concentration of acetaldehyde was higher than 10 mg/L, the yogurt had a typical fragrance, or when the ratio of the acetaldehyde content to the diacetyl content was greater than 3:1, the yogurt would have a characteristic flavor, and the higher the acetaldehyde content, the more obvious the characteristic flavor. The yogurt prepared from the compound microbial inoculum described in the present disclosure has both the typical fragrance and characteristic flavor.

TABLE 9
Determination results of acetaldehyde and
diacetyl in different combined yogurt

| Yogurt combination | Acetaldehyde | Diacetyl |
|---|---|---|
| 6-4 + D-2 + Lacticaseibacillus rhamnosus | 17.6 ± 0.003 | 5.65 ± 0.04 |
| FMBL L23004 CNN + Lactiplantibacillus | | |
| plantarum FMBL L23036 CNN | | |
| 6-4 + D-2 | 14.08 ± 0.719 | 3.54 ± 0.02 |
| P-value | <0.01 | <0.01 |

(3) Determination of yogurt texture

The yogurt prepared from the compound microbial inoculum described in the present disclosure is higher in hardness and viscosity compared with the control group, with better rheological properties. The yogurt prepared from the compound microbial inoculum has higher cohesion and a good degree of aggregation inside the yogurt. The viscosity index of the yogurt prepared from the compound microbial inoculum is higher and less affected by temperature. The texture characteristics of the yogurt prepared from the compound microbial inoculum are better compared with the control group.

TABLE 10
Determination results of texture characteristics of different combined yogurt

| | Texture characteristics | | | |
|---|---|---|---|---|
| | | | | Viscosity |
| | Hardness | Viscosity | Cohesion | index |
| Yogurt combination | (g) | (g · s) | (g) | (g · s) |
| 6-4 + D-2 + Lacticaseibacillus | 104.10 ± 3.92 | 786.94 ± 5.43 | −61.25 ± 1.92 | −288.89 ± 4.35 |
| rhamnosus FMBL L23004 | | | | |
| CNN + Lactiplantibacillus | | | | |
| plantarum FMBL L23036 | | | | |
| CNN | | | | |
| 6-4 + D-2 | 50.78 ± 1.65 | 363.13 ± 8.78 | −42.44 ± 2.34 | −61.25 ± 2.71 |
| P-value | <0.01 | <0.01 | <0.01 | <0.01 |

### (4) Determination of the Number of Lactic Acid Bacteria in Yogurt

A. Determination of the number of lactic acid bacteria in yogurt

In this study, an M17 medium (S. thermophilus) and an MRS medium (lactic acid bacteria) were used to count the total amount of S. thermophilus and lactic acid bacteria in yogurt.

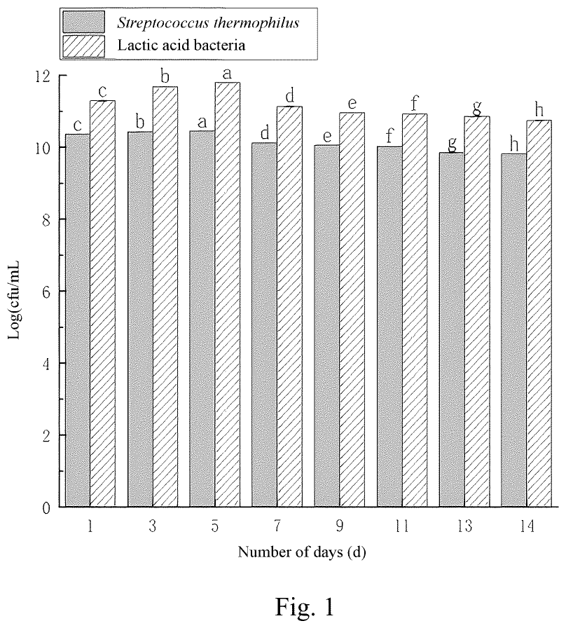

As shown in FIG. 1, the number of viable bacteria of S. thermophilus and lactic acid bacteria showed a trend of first increasing and then decreasing with the increase of storage time. However, after 14 days of storage, it was still greater than 10⁷ CFU/mL, which was in line with the national standard.

### B. Real-Time Fluorescence Quantitative PCR

Through a real-time fluorescence quantitative PCR reaction, standard curves for bacterial count determination of Lacticaseibacillus rhamnosus FMBL L23004 CNN and Lactiplantibacillus plantarum FMBL L23036 CNN were established.

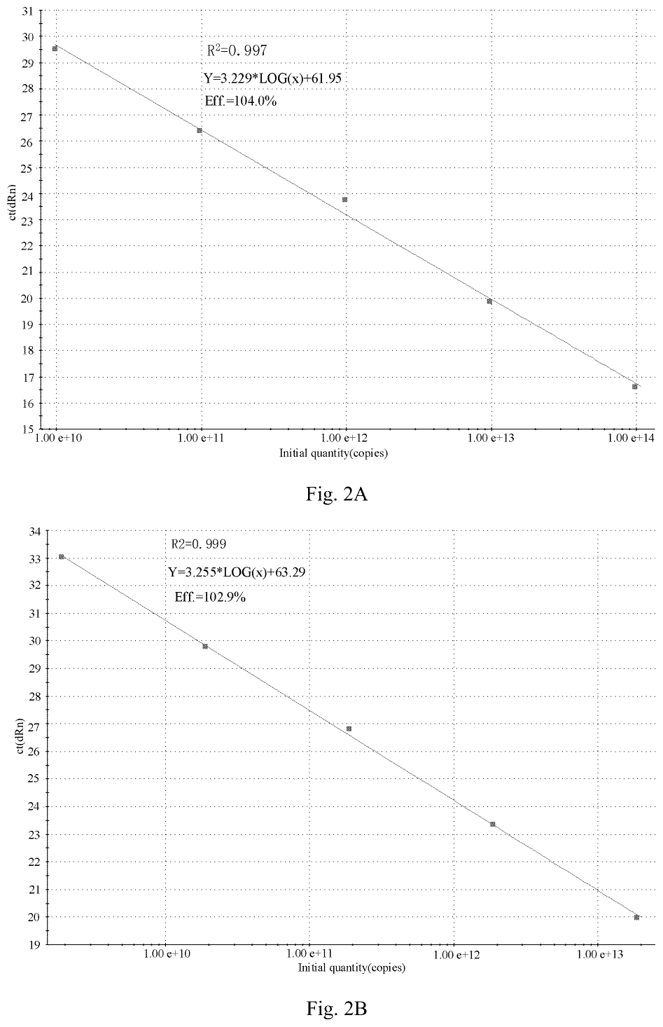

As shown in FIG. 2A and FIG. 2B, the results of fluorescence quantitative PCR amplification of the gradient diluted DNA liquid are obvious in discriminability. Standard curves for counting Lacticaseibacillus rhamnosus FMBL L23004 CNN and Lactiplantibacillus plantarum FMBL L23036 CNN were automatically drawn by software according to the number of bacteria and the Ct value. Through calculation, it could be seen that R² of the standard curve of Lactiplantibacillus plantarum FMBL L23036 CNN was greater than 0.99, and the amplification efficiency of the standard curve was 102.9%; R² of the standard curve of Lacticaseibacillus rhamnosus FMBL L23004 CNN was greater than 0.99, and the amplification efficiency of the standard curve was 104%; the closer R² was to 1, the better the linear correlation, and the ideal range of the amplification efficiency of the standard curve was within 90%-110%. The amplification efficiency in this experiment was within this range, which could better meet the basic experimental requirements of fluorescence quantitative PCR in this study.

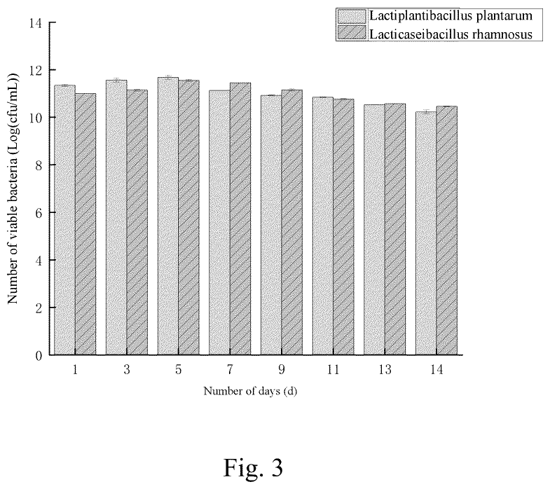

According to the standard curves of Lacticaseibacillus rhamnosus FMBL L23004 CNN and Lactiplantibacillus plantarum FMBL L23036 CNN, the numbers of viable bacteria of Lactiplantibacillus plantarum FMBL L23036 CNN and Lacticaseibacillus rhamnosus FMBL L23004 CNN in yogurt DNA samples stored at different time were calculated. The results are shown in FIG. 3, and it can be seen that after 5 days of storage, the numbers of viable bacteria of Lactiplantibacillus plantarum FMBL L23036 CNN and Lacticaseibacillus rhamnosus FMBL L23004 CNN gradually decrease with the increase of storage time, but remain above 10⁷ CFU/mL at 14 days, indicating that Lactiplantibacillus plantarum FMBL L23036 CNN and Lacticaseibacillus rhamnosus FMBL L23004 CNN can survive well in yogurt and play a better role, which meets the national standards.

### (5) Determination of Flavor Substances in Yogurt

Through the determination of volatile flavor substances in two samples, it can be found that 40 volatile flavor substances were detected, including alcohols, acids, aldehydes, ketones, esters and other types. In control group samples, 26 volatile flavor substances were detected, including two alcohols, eight acids, one ester, one aldehyde, eight ketones and six other types. In samples to be tested, 28 volatile flavor substances were detected, including six alcohols, five acids, three esters, two aldehydes, seven ketones and five other types.

TABLE 11
Determination results of volatile flavor substances

| | | | | Mass concentration | |
|---|---|---|---|---|---|
| | | | | (μg/mL) | |
| | | Retention | Volatile flavor | Control | |
| Item | No. | time/min | substance | group | Sample |
| Alcohols | 1 | 13.86 | 2-butyl-1-octanol | 0.026 | 0 |
| | 2 | 8.40 | 1-hexanol | 0 | 0.235 |
| | 3 | 9.73 | 3, 5-octadiene-2-ol | 0 | 0.01 |
| | 4 | 9.46 | 1-octen-3-ol | 0 | 0.013 |
| | 5 | 9.37 | 1-heptanol | 0 | 0.083 |
| | 6 | 11.30 | 1-nonanol | 0 | 0.029 |
| | 7 | 36.05 | Octaethylene glycol | 0.01 | 0.030 |
| | | | monododecyl ether | | |
| Acids | 8 | 5.52 | Acetic acid | 0.057 | 0.075 |
| | 9 | 7.47 | Butyric acid | 0.041 | 0.099 |
| | 10 | 9.40 | Caproic acid | 0.13 | 0.361 |
| | 11 | 11.34 | Caprylic acid | 0.044 | 0.079 |
| | 12 | 13.30 | n-capric acid | 0.01 | 0 |
| | 13 | 17.23 | Myristic acid | 0.039 | 0 |
| | 14 | 19.20 | n-palmitic acid | 0.184 | 0.067 |
| | 15 | 16.84 | 3-hydroxylauric acid | 0.067 | 0 |
| Esters | 16 | 5.72 | Vinyl formate | 0.031 | 0.187 |
| | 17 | 5.83 | Ethyl acetate | 0 | 0.039 |
| | 18 | 11.54 | Octyl formate | 0 | 0.080 |
| Aldehydes | 19 | 11.84 | 2-decenal, (E)- | 0 | 0.008 |
| | 20 | 19.43 | Aldehyde | 0.150 | 0.170 |
| Ketones | 21 | 5.52 | 2-butanone | 0.032 | 0 |
| | 22 | 6.87 | 2,3-butanedione | 0.141 | 0 |
| | 23 | 6.83 | 2-methyl-3- | 0.049 | 0 |
| | | | pentanone | | |
| | 24 | 8.75 | 2-methyl-3- | 0.2 | 0.2 |
| | | | heptanone | | |
| | 25 | 8.39 | 2-heptanone | 0.157 | 0.117 |
| | 26 | 10.31 | 2-nonanone | 0.053 | 0.041 |
| | 27 | 6.99 | Acetoin | 0.223 | 0.530 |
| | 28 | 12.24 | 2-undecanone | 0.012 | 0.013 |
| | 29 | 7.83 | 2,3-pentanedione | 0 | 0.086 |
| | 30 | 13.63 | 2H-pyran-2-one | 0 | 0.010 |
| | | | tetrahydro-6-pentyl | | |
| Other | 31 | 13.23 | 1-hexoxy-3- | 0.031 | 0 |
| types | | | methylhexane | | |
| | 32 | 6.86 | 2,4-dimethylhexane | 0.016 | 0 |
| | 33 | 8.05 | 1-octene | 0.034 | 0 |
| | 34 | 6.73 | 1-methoxy-2- | 0 | 0.036 |
| | | | propylamine | | |
| | 35 | 6.28 | 2-ethyloxetane | 0 | 0.037 |
| | 36 | 8.14 | Octane | 0 | 0.052 |
| | 37 | 6.68 | 2-(1, | 0 | 0.063 |
| | | | 1-dimethylethyl)-3- | | |
| | | | methylepoxyethane | | |
| | 38 | 5.98 | Oxirane | 0.010 | 0.020 |
| | 39 | 9.94 | 5-ethyl-2, 2, | 0.016 | 0 |
| | | | 3-trimethylheptane | | |
| | 40 | 9.81 | 6-ethyl-2- | 0.051 | 0 |
| | | | methyloctane | | |

The detection results are shown in Table 11, and it can be seen that a total of seven alcohol flavor substances were detected, where 2-butyl-1-octanol was only detected in the control group, while six alcohol substances were detected in the samples, including 1-hexanol, 3, 5-octadiene-2-ol, 1-octen-3-ol, 1-heptanol, 1-nonanol and octaethylene glycol monododecyl ether. The presence of these alcohols contributes to the good flavor of yogurt, and the alcohols can also be used as nutrients for lactic acid bacteria to promote the reproduction of lactic acid bacteria, so that the taste and quality of yogurt are improved.

As can be seen from Table 11, eight acid flavor substances were detected, where eight acid flavor substances were found in the control group without their unique acid substances, while five acid flavor substances were detected in the samples without their unique substances. Acetic acid, butyric acid, caproic acid, caprylic acid and n-palmitic acid are common acids in the two yogurt samples, and they play an important role in the flavor of yogurt; and acetic acid and caproic acid are the main reasons for the sour taste of yogurt, and caproic acid also increases the smell of yogurt and helps to produce floral flavor. Lactic acid plays an important role in refreshing sour taste of yogurt, but because of its low volatility, it was not detected in the two yogurt samples. It can be said that these acids have a significant impact on the flavor and aroma of yogurt.

From Table 11, it can be seen that a total of three ester flavor substances were detected, where three were detected in the samples. Ethyl acetate and octyl formate were unique to the samples to be tested; and there was one ester substance detected in the control group, and there were no unique esters. Only two aldehydes were detected in the yogurt prepared from the compound microbial inoculum, with 2-decenal being a unique aldehyde. These esters can improve the taste and flavor of yogurt.

According to Table 11, a total of 10 ketone flavor substances were detected, where 7 ketone flavor substances were detected in the samples to be tested, with 2, 3-pentanedione and 2H-pyran-2-one tetrahydro-6-pentyl being unique; and there were 8 in the control group, where 2-butanone, 2, 3-butanedione, and 2-methyl-3-pentanone were unique. Acetoin has a mild creamy flavor, and is slightly sweet and similar to the taste of butter. It is a common flavor substance in dairy products, and has a significant impact on flavor. Diacetyl is an important aromatic compound that gives a butter flavor, and a small amount of diacetyl helps to form the unique flavor and aroma of yogurt. In addition, the combination of diacetyl and acetoin brings a mild, pleasant, and buttery taste, which is crucial for the taste of yogurt.

In summary, the present disclosure provides a compound microbial inoculum with bacteriostatic effect. The compound microbial inoculum includes Lacticaseibacillus rhamnosus FMBL L23004 CNN and Lactiplantibacillus plantarum FMBL L23036 CNN. The compound microbial inoculum has the effect of preventing and/or treating diarrhea, and the compound microbial inoculum also has antibiotic sensitivity; as a yogurt starter, the compound microbial inoculum allows yogurt obtained by fermentation to have suitable acidity and higher water holding capacity, as well as better texture characteristics, and have the typical flavor characteristics of yogurt; and the yogurt fermented by the compound microbial inoculum has higher probiotic activity.

What is claimed is:

1. A compound microbial inoculum with bacteriostatic effect, wherein the compound microbial inoculum comprises Lacticaseibacillus rhamnosus FMBL L23004 CNN and Lactiplantibacillus plantarum FMBL L23036 CNN, wherein the Lacticaseibacillus rhamnosus FMBL L23004 CNN was deposited on 26 Jun. 2023 at China Center for Type Culture Collection (CCTCC) under accession number CCTCC NO: M 20231099; and the Lactiplantibacillus plantarum FMBL L23036 CNN was deposited on 26 Jun. 2023 at CCTCC under accession number CCTCC NO: M 20231101.

2. Use of the compound microbial inoculum according to claim 1 in the preparation of drugs for inhibiting pathogenic bacteria.

3. The Use according to claim 2, wherein the pathogenic bacteria comprise one or more of Escherichia coil EPEC, Escherichia coil ETEC, Salmonella enterica subsp. enterica serovar Typhimurium, Escherichia coil EHEC, Listeria monocytogenes, and Salmonella enterica subsp. Enterica.

4. Use of the compound microbial inoculum according to claim 1 in the preparation of drugs for preventing and/or treating diarrhea.

5. Use of the compound microbial inoculum according to claim 1 in the preparation of food, food additives or health care products.

6. Use of the compound microbial inoculum according to claim 1 in the preparation of yogurt or yogurt starter.

7. Yogurt fermented by the compound microbial inoculum according to claim 1.
